(19) United States
(12) Patent Application Publication
(10) Pub. No.: US 2009/0171376 A1
(43) Pub. Date: Jul. 2, 2009

(54) Mesh Anchoring System

(76) Inventors: Allen Burton, Houston, TX (US); Phillip C. Phan, Houston, TX (US)

Correspondence Address: FULBRIGHT & JAWORSKI L.L.P., 600 CONGRESS AVE., SUITE 2400, AUSTIN, TX, 78701, US

(21) Appl. No.: 12/092,804

(22) PCT Filed: Nov. 7, 2006
(86) PCT No.: PCT/US06/43371

§ 371 Date: Sep. 10, 2008

Related U.S. Application Data
(63) Continuation of application No. 60/734,152, filed on Nov. 7, 2005.

(51) Int. Cl. A61B 17/08

(52) U.S. Cl. 606/151

(57) ABSTRACT

Medical devices configured for securing the position of a tube or wire in a desired position. Embodiments of the medical devices include an anchoring element and a mesh element coupled to the anchoring element. The medical device can be secured to the skin surface of a subject, or to muscle or fascia within a surgical incision. The medical device can be secured, for example, by stapling the mesh element to underlying skin or tissue.

This application claims the benefit of priority to U.S. Provisional Patent Application Ser. No. 60/734,152, filed Nov. 7, 2005, hereby incorporated by reference in its entirety.

### BACKGROUND OF THE INVENTION

1. Field of the Invention

The present invention relates generally to anchoring devices. More specifically, the invention relates to anchoring devices configured to secure the placement of a medical element, such as a tube (e.g., a catheter) or wire (e.g., a simulator lead), on a body surface or within a body, such as in a surgical wound. The invention also relates to methods of anchoring a medical element on or within a body, to kits that include one or more of the present anchoring devices, and to methods of delivering a drug to a subject.

2. Description of Related Art

Treatment of severe pain in the seriously ill patient, whether cancer-related or not, remains a significant challenge in medicine today. Treatment of cancer pain and chronic pain depends initially on traditional pharmacotherapy, i.e., opioids and adjuvant medications. Some of these patients are refractory to pharmacological therapy, either from failure to provide analgesia or intolerable opioid-related side effects.

Many interventions are available to help treat patients with pain. Examples include placement of an intrathecal catheter with a pump system for delivery of analgesic medications, and a spinal cord stimulator system. Both of these systems act to modulate pain transmission at the spinal cord. An intrathecal pump infuses opioids to act on opioid receptors on the spinal cord and thus block pain of neuropathic origin, although the exact mechanism is still unclear.

Both the intrathecal pump and spinal cord stimulator require access to the intraspinal spaces. An intrathecal pump has its catheter placed directly within the intrathecal space, whereas the spinal cord stimulator has its leads paced within the epidural space. One of the biggest challenges of both systems is to maintain placement of the intraspinal catheter and stimulator leads by “anchoring” them in position following proper placement in their designated spaces. Lead migration is the most common complication of implanted spinal cord stimulators. Thus, it is essential that the stimulator lead with its many electrodes stays at its designated position so that the electrical fields created act on the relevant pain pathways.

Numerous intrathecal catheter and stimulator anchors are available. However, they generally require the physician to use suture material to secure the catheter or stimulator lead to surrounding tissue.

### SUMMARY OF THE INVENTION

The present invention provides for anchoring devices and methods that can secure a medical element such as a tube or a wire in a particular position on or within the body using non-suture materials, such as staples. The present medical devices may be sterilized for use during a medical procedure, and also may be sold in kits.

Embodiments of the present medical devices include an anchoring element and a mesh element coupled to the anchoring element. In particular embodiments, the anchoring element is a rigid anchoring element. “Rigid” does not mean inflexible. In particular embodiments, the medical device includes a rigid anchoring element and a mesh element coupled to the rigid anchoring element, the medical device being configured such that a linear portion of a medical element can be: (a) at least partially surrounded by the rigid anchoring element; (b) positioned between the rigid anchoring element and tissue; and (c) secured to tissue using one or more staples that penetrate the mesh element.

The rigid anchoring element and the mesh element can be coupled in any manner known to those of ordinary skill in the art. For example, the anchoring element and the mesh element can be coupled in close proximity to each other, coupled such that the mesh element extends from the anchoring element, coupled such that the anchoring element extends from the mesh element, coupled such that the mesh element is in direct contact with the anchoring element, or coupled such that the closest portion of the mesh element is within 1, 2, 5, 10, 15, 20, 25, 30, 35, 40, 45, 50, 55, 60, 65, 70, 75, 80, 85, 90, 95, 100, 120, 140, 160, 180, 200, 250, 300, 350, 400, 450, 500, 1000, 2000, 3000, 4000, or 5000 millimeters of the closest portion of the anchoring element. In other words, in some embodiments, the smallest gap between the two elements may be small in distance. In embodiments in which there is no intervening structure or portion of a patient between the region in which two elements are coupled (save any bonding agent that is used), the two elements may be characterized as being directly connected to each other.

In some embodiments, the rigid anchoring element includes a medical element-retaining section. The medical element-retaining section can be configured to receive a linear portion of a medical element or a non-linear portion of a medical element. The medical element-retaining section may be open along the length, or closed along the length. In particular embodiments, the medical element-retaining section has two ends and is open at each end.

In some embodiments, the rigid anchoring element further includes a first flange coupled to the medical element-retaining section. The medical-element retaining section and the first flange may be integrally formed or separately fabricated and then attached. The rigid anchoring element may further include a second flange coupled to the medical element-retaining section. The medical-element retaining section and the second flange may be integrally formed, or separately fabricated and then attached. The mesh element may include a first mesh element portion attached to the first flange and a second mesh element portion attached to the second flange. The mesh element may include a shell coupled to an outer surface of the rigid anchoring element, the mesh element including a first portion that extends beyond an end of the first flange and a second portion that extends beyond an end of the second flange.

In particular embodiments, as discussed in greater detail below, the mesh element encloses the rigid anchoring element. The mesh element and the rigid anchoring element may be integrally formed, or separately fabricated and then attached.

In some embodiments, the rigid anchoring element includes a first jaw portion and a second jaw portion connected to each other. The mesh element may extend from the first jaw portion or the second jaw portion. In further embodiments, the mesh element includes a first mesh element portion extending from the first jaw portion and a second mesh element portion extending from the second jaw portion. The first jaw portion and the second jaw portion may be connected to each other in any manner known to those of ordinary skill in the art. In particular embodiments, the first jaw portion is hinged to the second jaw portion at one location. In some embodiments, the rigid anchoring element is configured such that the two jaw portions can be interconnected together at a second location. In other embodiments, the medical device includes a first jaw element, a second jaw element connected to the first jaw element; a first mesh element coupled to the first jaw element; and a second mesh element coupled to the second jaw element. The first jaw element can be connected to the second jaw element in any manner known to those of ordinary skill in the art, such as by a hinge.

The mesh element can be composed of any material or combination of materials known to those of ordinary skill in the art. For example, the mesh element may include one or more materials selected from the group consisting of aluminum, titanium, steel, and nitinol. In some embodiments, the mesh element includes polypropylene. The mesh element may be flexible or rigid.

The mesh element can be fabricated in any manner known to those of ordinary skill in the art. In some embodiments, the mesh element includes a plurality of interwoven filaments. The filaments, for example, may be metallic filaments. In further embodiments, the filaments are biodegradable. The biodegradable material can be any material known to those of ordinary skill in the art. For example, the biodegradable material can include one or more polymers selected from the group consisting of a poly(dioxanone), a poly(trimethylene carbonate), a polycaprolactone, a polyanhydride, a polyorthoester, a polyphosphazene, a polyglycolic acid, a polydioxanone, polyhydroxybutyrate, and a polylactide. The filaments may or may not be affixed to each other at crossing locations. In still further embodiments, the mesh element includes a sheet having a plurality of openings. The openings can be any suitable size or of any suitable number.

The anchoring element can be composed of any material or combination of materials known to those of ordinary skill in the art. In some embodiments, the rigid anchoring element includes one or more materials selected from the group consisting of silicone and plastic. In some embodiments, the plastic includes one or more materials selected from the group consisting of polyethylene and polytetrafluoroethylene. In further embodiments, the plastic includes one or more materials selected from the group consisting of polyurethane, polypropylene, and polyimide.

In some embodiments, the medical device further includes a drug molecule attached to the mesh element. For example, in some embodiments, the mesh element is coated with a composition that includes a drug and a pharmaceutically acceptable carrier. In further embodiments, a composition that includes the drug is applied to one or more surfaces of the mesh element. In further embodiments, the drug is dispersed within the mesh element material. Thus, for example, the drug may be released from the mesh element following application/insertion of the medical device.

“Drug” is defined herein to refer to denote a compound that has beneficial therapeutic and/or prophylactic properties when administered to a subject. The term “drug” includes the neutral form of the drug, pharmaceutically acceptable salts, and prodrugs. In some embodiments, the drug is selected from the group consisting of an antimicrobial agent, a growth factor, and an anti-inflammatory agent. In some embodiments, the drug is an antimicrobial agent, such as an antibiotic, an antiviral agent, or an antifungal agent. In particular embodiments, the drug is an antibiotic selected from the group consisting of erythromycin, bacitracin, neomycin, penicillin, polymyxin B, tetracycline, viomycin, chloromycetin, streptomycin, cefazolin, ampicillin, azactam, tobramycin, clindamycin, and gentamycin. In further embodiments, the drug is a growth factor. For example, the growth factor may be Interleukin (IL)-1, IL-2, IL-4, IL-5, IL-6, IL-7, IL-9, IL-10, IL-13, IL-15, IL-16, IL-17, IL-18, IL-21, Ciliary Neurotrophic Factor (CNTF), erythropoietin, Vascular Endothelial Growth Factor (VEGF), angiopoietin 1 (Ang-1), angiopoietin 2 (Ang-2), TNF, Interferon-gamma, GM-CSF, TGFbeta, and TNF Receptor. Examples of anti-inflammatory agents include include betamethasone phosphate, dexamethasone phosphate, prednisolone phosphate, hydrocortisone phosphate, prednisolone succinate, hydrocortisone succinate, loxoprofen sodium, and diclofenac sodium. Examples of pharmaceutically acceptable carrier includes any substances which does not interfere with effectiveness of the drug that is not toxic to the subject to which it is administered. Examples of carriers include buffering agents, surfactants, preservatives, solubilizing agents, stabilizing agents, polymers, and/or pH-adjusting agents.

Once positioned in a desired location, the mesh element can be secured in position using non-suture material, such as a staple or staples that are placed using, for example, a staple gun. Using one or more staples to secure the mesh element may take less time and require a smaller incision than conventional suture techniques. A staple can be used to secure the mesh element to any extrabody or intrabody location, such as a skin surface or tissue (such as muscle or fascia) within a wound. When placed within a wound, for example, the body tissue will over time form an even stronger adhesion tissue layer around the mesh, thus serving as a second natural anchoring system in addition to the mesh anchor.

Embodiments of the present anchoring devices may be widely applied to any intrabody and extrabody medical element (such as a catheter, other tubing, or a lead of some kind) that requires a secure attachment to any body tissue. For example, embodiments of the present devices may be used to secure pacemaker electrodes, central lines, body cavity drains, chest tubes, or even urologic catheters. Other examples of medical elements that may be secured using embodiments of the present anchoring devices include pacemaker wires, ventricular (neurological) access catheters and other percutaneously inserted lines (including central venous access catheters), nephrostomy tubes, biliary drains, chest drainage catheters, as well as temporary vascular access devices used in interventional cardiology and radiology procedures.

Another embodiment of the present invention concerns a kit that includes (a) a medical device that includes a rigid anchoring element and a mesh element coupled to the anchoring element; and (b) a tube or wire. The medical device can be any of those devices discussed above. For example, the tube may be a catheter, a chest tube, or a urological catheter. The catheter may be any catheter known to those of ordinary skill in the art. In particular embodiments, the catheter is an intrathecal catheter. Similarly, the wire can be any wire known to those of ordinary skill in the art, such as a stimulator lead or a pacemaker electrode. In some embodiments, the kit includes a medical device that has a drug attached to the mesh element. The drug can be any of those drugs set forth above, and the drug can be attached to the mesh element in any manner as set forth above.

Embodiments of the present methods include securing one of the present medical devices to a living being at a target location. In some embodiments, the target location is subcutaneous. In other embodiments, the target location is above the skin.

The present invention also generally pertains to a method of delivering a drug to a subject, involving contacting the subject with a medical device that includes a rigid anchoring element, a mesh element coupled to the rigid anchoring element, and a drug in contact with the mesh element, wherein the drug is delivered to the subject. The medical device can be any of those medical devices set forth above that include a drug in contact with the mesh element.

The subject can be any subject, such as a mammal. In particular embodiments, the subject is a human. In more particular embodiments, the subject is a patient with chronic pain.

Other embodiments of the present medical devices, kits and methods, and details associated with those embodiments and the ones listed above, are provided below.

### BRIEF DESCRIPTION OF THE DRAWINGS

The following drawings illustrate by way of example and not limitation.

FIG. 1 shows an embodiment of one of the present medical devices.

FIG. 2 shows an embodiment of one of the present medical devices that does not include a flange.

FIG. 3A shows an embodiment of one of the present medical devices in the open position.

FIG. 3B shows the medical device of FIG. 3A in the closed position with a linear portion of a medical element (e.g., catheter) in place.

FIG. 4 shows an embodiment of one of the present medical devices that is configured to receive a non-linear portion of a medical element.

FIG. 5 shows an embodiment of one of the present medical devices that is configured to receive a non-linear portion a medical element. In this embodiment, flanges extend outwardly from the anchoring element, and a mesh element portion extends outwardly from each flange.

FIG. 6A shows an embodiment of one of the present medical devices in which the mesh element is a shell coupled to an outer surface of the anchoring element

FIG. 6B shows an embodiment of one of the present medical devices in which the mesh element encloses the anchoring element.

FIG. 7 shows an embodiment of one of the present medical devices in which the anchoring element includes first and second jaw portions connected to each other.

FIG. 8 shows an embodiment of one of the present medical devices in which the anchoring element includes first and second jaw portions that are configured to latch together.

FIG. 9 shows an embodiment of one of the present medical devices in which the anchoring element is configured as two separate pieces that are attached together. A mesh element portion extends from each piece of the anchoring element.

FIG. 10 shows an embodiment of one of the present kits that includes a sterilized version of one of the present medical devices, a catheter, instructions for use (IFU), and a sealable package.

### DESCRIPTION OF ILLUSTRATIVE EMBODIMENTS

The terms “comprise” (and any form of comprise, such as “comprises” and “comprising”), “have” (and any form of have, such as “has” and “having”), and “include” (and any form of include, such as “includes” and “including”) are open-ended linking verbs. As a result, a device or method that “comprises,” “has,” or “includes” one or more elements possesses those one or more elements, but is not limited to possessing only those one or more elements. Likewise, an element of a device or a step of a method that “comprises,” “has,” or “includes” one or more features possesses those one or more features, but is not limited to possessing only those one or more features. Furthermore, a device or structure that is configured in a certain way is configured in at least that way, but may also be configured in ways that are not listed.

Thus, and by way of example, a medical device “comprising” a rigid anchoring element and a mesh element coupled to the rigid anchoring element is a medical device that has, but is not limited to having only, the recited features. That is, the medical device possesses at least the recited features, but does not exclude other features that are not expressly recited.

The terms “a” and “an” are defined as one or more than one unless this disclosure explicitly requires otherwise. The term “substantially” is defined as at least close to (an includes) a given value or state preferably within 10% of, more preferably within 1% of, and most preferably within 0.1% of).

Some embodiments of the present medical devices include a rigid anchoring element and a mesh element coupled to the rigid anchoring element. An “anchoring element” is an element that is configured to affix or secure a second element using one or more fasteners. The second element may be a medical element such as a catheter, wire, stent, tubing, or the like. A “rigid anchoring element” is an anchoring element that, by virtue of its composition and/or structure, exhibits a resistance (though not necessarily a complete resistance) to a change in its shape such as through bending or the like.

A “mesh element” is defined as an element that has two or more openings. Some embodiments of the present mesh elements include multiple mesh element portions that are separate from each other.

The rigid anchoring element may be coupled to the mesh element by any manner known to those of ordinary skill in the art. In some embodiments, the mesh element and the rigid anchoring element are in close proximity to each other, coupled such that the mesh element extends from the anchoring element, coupled such that the anchoring element extends from the mesh element, coupled such that the mesh element is in direct contact with the anchoring element, or coupled such that the closest portion of the mesh element is within 1, 2, 5, 10, 15, 20, 25, 30, 35, 40, 45, 50, 55, 60, 65, 70, 75, 80, 85, 90, 95, 100, 120, 140, 160, 180, 200, 250, 300, 350, 400, 450, 500, 1000, 2000, 3000, 4000, or 5000 millimeters of the closest portion of the anchoring element.

Embodiments of the present medical devices may be used to secure a medical element, such as a catheter or wire, in a particular location. For example, a spinal cord stimulator lead may be anchored using an embodiment of the present medical devices in a position such that the lead remains in the epidural space.

The mesh element of some embodiments of the present medical devices may be anchored to underlying or surrounding tissue by any method known to those of ordinary skill in the art. For example, in some embodiments, the mesh element may be stapled into place using any suitable stapling device. More specifically, the mesh element may be stapled to tissue within the body, such as tissue that is accessed through a surgical incision. Alternatively, the mesh element may be stapled to a skin surface, such as to secure the placement of a chest tube or wound drain. In some embodiments, the mesh element may comprise what is known in the art as surgical mesh, and may (in the same or other embodiments) be impregnated with an antimicrobial or antimicrobials, a growth factor or growth factors, etc., as desired.

The rigid anchoring element of some embodiments of the present medical devices may have any suitable configuration. The configuration will be largely dictated by the medical element to be secured. For example, the rigid anchoring element may be configured to include a medical element-retaining section that is cylindrical in shape and that has an opening along its length sized to accommodate a medical element such as a catheter in one embodiment and a wire in another. The present medical devices also may include additional features, such as an element having an opening sized to accept a suture.

The rigid anchoring element of some embodiments of the present medical devices may be composed of any material or combination of materials known to those of ordinary skill in the art that yield a rigid structure. Examples of such materials include silicone, plastic, and a combination of silicone and plastic. Plastics are well-known to those of ordinary skill in the art. Examples of plastics include polymers such as polyurethane, polyethylene, polytetrafluoroethylene, polypropylene, polyimide, and derivatives of these polymers. In some embodiments, the rigid anchoring element may be composed of one or more suitable metals, such as titanium, aluminum, steel, and any suitable nickel-titanium alloy (e.g., nitinol). In still other embodiments, embodiments of the present rigid anchoring elements may comprise both metal and synthetic material, such as a metal core surrounded by (or embedded in) a plastic exterior.

The mesh element of some embodiments of the present medical devices may or may not be rigid. It may be composed of the same material as the anchoring element, or it may be composed of a different material or combination of materials. Broadly, the mesh element may be composed of any suitable material or combination of suitable materials known to those of ordinary skill in the art. In some embodiments, the mesh element may comprise a shape-memory material, such as nitinol. In other embodiments, the mesh element comprises one of the other metals set forth above. In other embodiments, the mesh element may comprise polypropylene. In still other embodiments, the mesh element may comprise a biodegradable material. Examples of biodegradable materials include polymers such as poly(dioxanone), a poly(trimethylene carbonate), a polycaprolactone, a polyanhydrides, a polyorthoester, a polyphosphazene, a polyglycolic acid, a polydioxanone, polyhydroxybutyrate, and a polylactide. Following implantation of embodiments of the present medical devices into tissue, it is anticipated that the physiological response would include scar tissue formation around and within the mesh element, such that the end result would be an even stronger attachment than using attachment elements such as staples alone.

Medical Devices

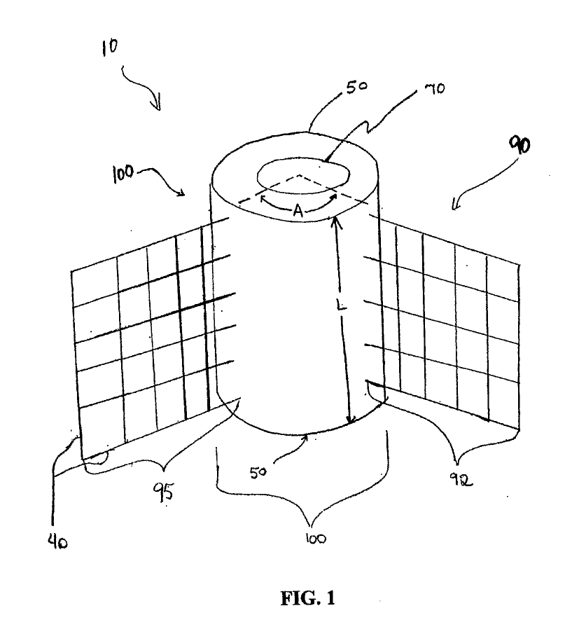

FIG. 1 shows medical device 10, which is one embodiment of one of the present medical devices. Medical device 10 includes anchoring element 100 coupled to mesh element 90. Mesh element 90 includes a first mesh element portion 92 and second mesh element portion 95. The mesh elements are attached to anchoring element 100 and extend from anchoring element 100 at different locations along anchoring element 100; those locations may be separated by any suitable angle A chosen in view of the application for the medical device, as is true of any of the present medical devices that include mesh element portions. Furthermore, each mesh element portion may extend from the anchoring element at any suitable angle chosen in view of the application for the medical device, and the two portions may extend from the anchoring element at the same or different angles, as is true of any of the present medical devices that include mesh element portions.

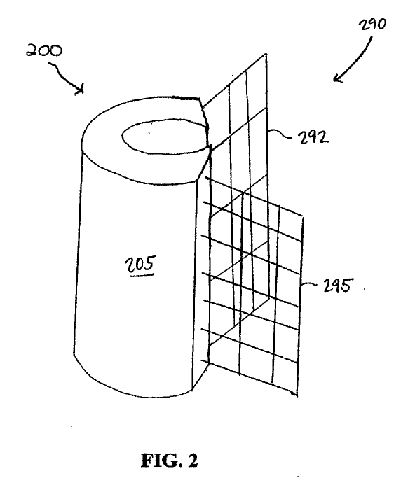

In the FIG. 1 embodiment, anchoring element 100 comprises a medical element-retaining section that has the form of a shaft having a passageway 70. The shaft is open at both its ends 50 and is closed along its length L, in contrast to the anchoring element of medical device 200 shown in FIG. 2. The shaft is configured to receive a linear portion of a medical element, such as a catheter, a drain, a tube, a wire, or a shunt, which may be passed through both ends 50.

As with each of the present anchoring elements, anchoring element 100 of medical device 10 may be rigid in some embodiments, and not rigid in other embodiments.

First mesh element portion 92 and second mesh element portion 95 are identical in size and configuration. In other embodiments, however, first mesh element portion 92 and second mesh element portion 95 are not identical in size and/or in configuration. For example, first mesh element portion 92 may be larger than second mesh element portion 95.

In the embodiment shown in FIG. 1, passageway 70 has a uniform diameter. In other embodiments, however, the passageway may have a non-uniform diameter to enable it to be grip a given medical element. For example, in some embodiments, passageway 70 may increase in size (e.g., diameter) from one end 50 to the other. The exterior of anchoring element 100 may have any suitable configuration. For example, while shown as a having a cylindrical exterior in the FIG. 1 embodiment, in other embodiments, anchoring element 100 may have a flat portion suited to contacting a flat part of the anatomy.

In some embodiments, mesh element 90 comprises a plurality of interwoven filaments 40. Filaments 40 may or may not be affixed to each other at crossing locations. In other embodiments, mesh element 90 comprises a sheet having a plurality of openings. The openings can be of any suitable size and configuration, and can be arranged in any suitable pattern.

Mesh element 90 may be flexible in some embodiments and inflexible in others. In some of the flexible embodiments, mesh element 90 may comprise material having shape memory, such as nitinol.

Mesh element 90 may be secured to body tissue with one or more staples placed using any suitable stapling device known to those of ordinary skill in the art. Mesh element 90 also may be secured to body tissue by any other method known to those of ordinary skill in the art, such as by suturing or tacking. Thus, in some embodiments, mesh element 90 includes one or more openings that are of sufficient size to accommodate the passage of a suture or a tack. Alternatively, mesh element 90 may be both sutured and stapled into position. In some embodiments, mesh element 90 also may include an integral tack or tacks (not shown) suitable for inserting into tissue such as muscle or deep fascia. In some embodiments, medical device 10 may include one or more attachment elements, such as suture, staple(s), or tack(s) that are coupled to mesh element 90 in such a way as to make the medical device easy to secure to a target location.

Medical device 10 may be fabricated by attaching mesh element portions 92, 95 to anchoring element 100 using any suitable means, such as heat, an adhesive, or the like, or by integrally forming the mesh and anchoring elements together as a single unit. As another alternative, mesh element portions 92, 95 may be embedded into anchoring element 100 in a standard molding procedure.

The present medical devices may include a mesh element that comprises more than two mesh element portions. For example, 3, 4, 5 or more mesh element portions (that are separate from each other) may be coupled to a given anchoring element if it best suits a given application. Such medical devices may be secured to a target location using one, some or all of the mesh element portions.

FIG. 2 shows medical device 200, which is another embodiment of the present medical devices. Medical device 200 includes anchoring element 205, which is coupled to mesh element 290. Mesh element 290 includes mesh element portions 292, 295, which may be coupled to anchoring element 205 in the same way that mesh element portions 92, 95 may be coupled to anchoring element 100, and may be made from the same materials and have the same configuration as mesh element portions 92, 95 of medical device 10. In this embodiment, anchoring element 205 is open along its length and at its ends. Anchoring element 205 comprises a medical element-retaining section that is shaped like a cylinder having a slit along its length, and is configured to receive a linear portion of a medical element. Anchoring element 205 may be clipped onto the linear portion of the medical element, and mesh element 290 may be attached to a living being at a target location in the manner described above. Alternatively, a linear portion of a medical element may be passed through the open ends of anchoring element 205. In embodiments of medical device 200 that include rigid versions of anchoring element 205, the anchoring element would still have sufficient flexibility to be able to clip onto a linear portion of a medical element, as is true of other similarly shaped anchoring elements. In other embodiments, anchoring element 205 is not rigid.

As with all of the present anchoring elements, anchoring element 205 and mesh element 290 can each have any suitable size, which will be largely dependent on the medical element to be secured at a target location. For example, for linear medical elements such as catheters, the mesh element may have the same length as the anchoring element, or it may be shorter than the anchoring element.

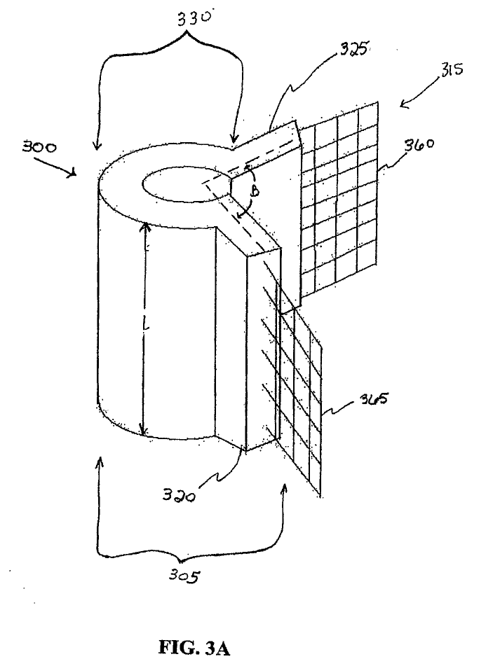

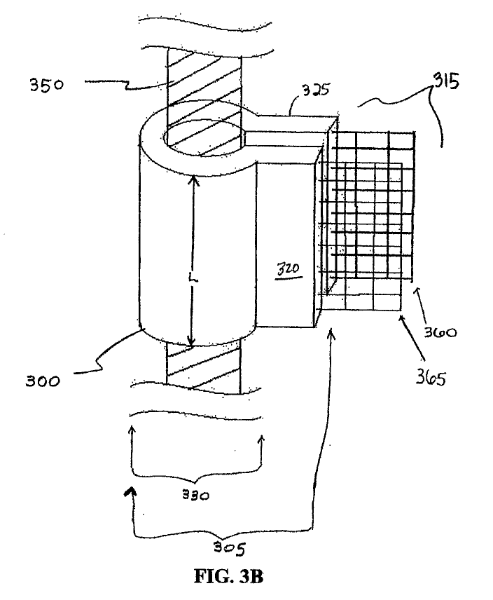

FIG. 3A shows medical device 300, which is another embodiment of the present medical devices. Medical device 300 includes anchoring element 300, which is coupled to mesh element 315. As with anchoring element 205 of medical device 200, anchoring element 305 of medical device 300 is open along length L. Anchoring element 305 is configured to receive a linear portion of a medical element, and is shown in an open position, unattached to a medical element. Anchoring element 305 comprises a medical element-retaining section 330 that is coupled to first flange 320 and second flange 325.

First flange 320 and second flange 325 are identical in size and configuration in the depicted embodiment. In other embodiments, however, first flange 320 may have a different size and/or configuration than second flange 325, especially if it renders medical device 300 better suited for a given application. The flanges may be integrally formed with the medical element-retaining section (as shown), or they may be attached to the medical element-retaining section using any suitable attachment method. The angle B that separates the flanges may be any suitable angle chosen in view of the application for the medical device, as is true of any of the present medical devices that includes flanges. Other embodiments of the present medical devices include a single flange, or more than two flanges, such as 3, 4, or 5 flanges. The number of flanges that are used will largely be dependent on the application for the medical device. Each of the flanges of any of the present anchoring elements, as well as any other suitable portion of any of the present anchoring elements, (such as one or both of the flanges of medical device 300) may include any number of openings, such as openings suitable for the optional passage of a suture.

Mesh element 315 includes first mesh element portion 365 attached to first flange 320, and second mesh element portion 360 attached to second flange 325. These mesh element portions may be coupled to anchoring element 305 in the same way that mesh element portions 92, 95 may be coupled to anchoring element 100, and may be made from the same materials and have the same configuration as mesh element portions 92, 95 of medical device 10.

FIG. 3B depicts medical device 300 in a closed position, following placement of a linear portion of medical element 350 in the medical element-retaining section 330. In this embodiment, anchoring element 305 clips onto medical element 350. Mesh element 315 can then be secured into position within the body or on a body surface using any of the attachment techniques (e.g., stapling) described above. With this and other embodiments of the present medical devices, the mesh element portions can be secured as a single unit within the body or on a body surface (e.g., using the same staple or staples to penetrate both mesh element portions), or each mesh element portion can be separately secured. Mesh element 315, and therefore mesh element portions 365 and 360, may be flexible enough for the mesh element portions to be bent (e.g., outwardly). In other embodiments, mesh element 315 may be inflexible.

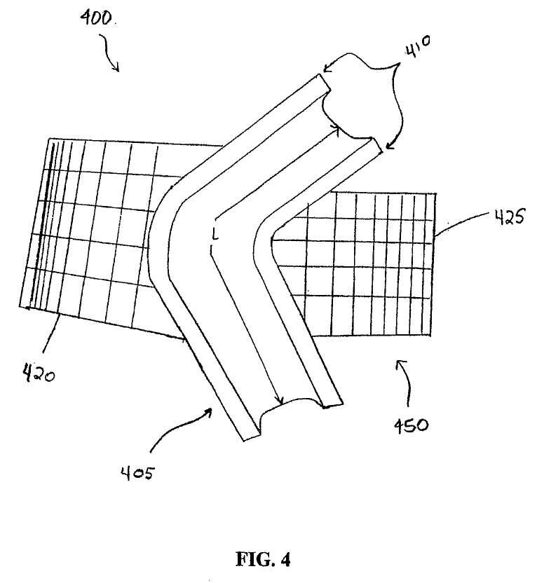

FIG. 4 depicts medical device 400, another embodiment of the present medical devices. Medical device 400 includes anchoring element 405, which is coupled to mesh element 450. Anchoring element 405 includes medical element-retaining section 410, which is shaped like an open channel. Medical element-retaining section 410 is open along its length L, although in other embodiment it is closed along its length. Medical element-retaining section 410 has a bend in it, and is therefore configured to receive a non-linear portion of a medical element. The non-linear portion of the medical element may be a flexible portion of a catheter or wire, or a rigid non-linear tubular element.

Mesh element 450 includes first mesh element portion 420 and second mesh element portion 425, which may be coupled to sides of medical element retaining section 410 using any of the techniques described above. Mesh element portions 420 and 425 may have any suitable configuration, and may be made from any suitable material.

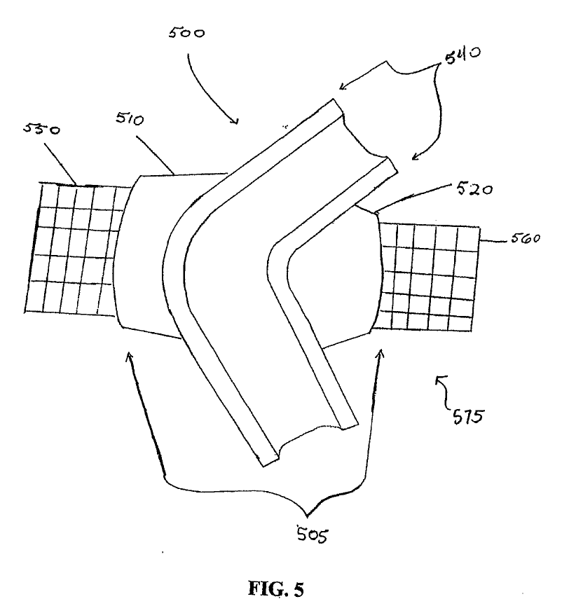

FIG. 5 shows medical device 500, which is similar to medical device 400. Medical device 500 includes anchoring element 505, which is coupled to mesh element 575. Anchoring element 505 includes first flange 510 and second flange 520, both of which are coupled to medical element-retaining section 540. The two flanges may have any suitable shape, and may be made from any suitable material. The coupling of the flanges to the medical element-retaining section may be achieved through, e.g., attachment or integral formation. Medical element-retaining section 540 is bent and therefore configured to receive a non-linear portion of a medical element. Mesh element 575 includes first mesh element portion 550 and second mesh element portion 560, which may be coupled to first and second flanges 510 and 520, respectively, using any of the techniques described above. Mesh element portions 510 and 520 may have any suitable configuration, and may be made from any suitable material.

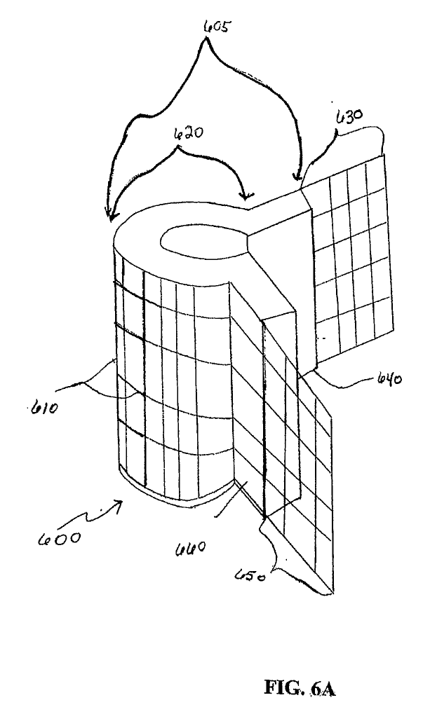

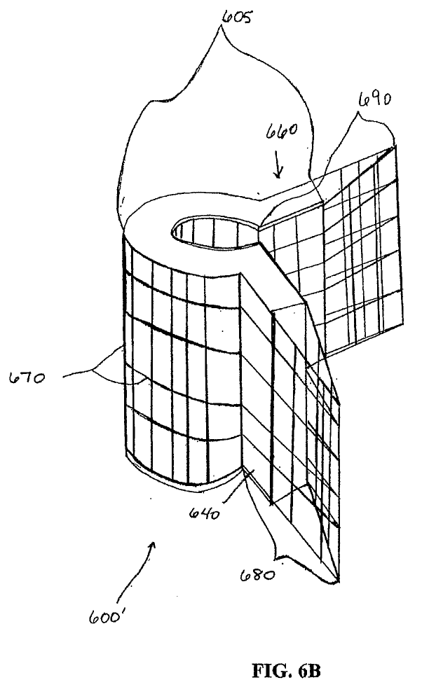

FIG. 6A depicts medical device 600 that include mesh element 610 coupled to anchoring element 605. More specifically, in this embodiment mesh element 610 comprises a shell that is coupled to an outer surface of anchoring element 605. Anchoring element 605 is similar to anchoring element 305 from FIG. 3A, and includes medical element-retaining section 620, which is open along its length and configured to receive a linear portion of a medical device, such as a catheter, wire, or other tube. First and second flanges 640 and 660, respectively, may be coupled to medical element-retaining section 620 as described above with respect to medical device 300, and may have any suitable shape and be made from any suitable material. Mesh element 610 includes a first mesh element portion 630 that extends beyond an end (or edge) of first flange 640 and a second mesh element portion 650 that extends beyond an end (or edge) of second flange 660. Mesh element 610 may have any suitable shape, and may be made of any suitable material.

The coupling of the mesh element to the anchoring element may be achieved through, e.g., attachment or integral formation. For example, mesh element 610 and anchoring element 605 may be separate initially, such that mesh element 610 may be snapped or folded onto anchoring element 605 following attachment of anchoring element 605 to a linear portion of a medical element. Mesh element 610 and/or anchoring element 605 may have any configuration suitable for coupling the two together in that manner, such as providing mesh element 610 with foldable tabs and/or providing anchoring element 605 with notches into which the tabs may be folded. Alternatively, where mesh element 610 is coupled to an anchoring element 605 that is sold separately, and where the size of the anchoring element is known, mesh element may be made from a shape memory material and treated to have a configuration that is suited to fitting around an outer surface portion of the anchoring element. The mesh element may then be deformed or “stretched out” and allowed to spring back to its initial configuration after positioning it to be coupled to the anchoring element. Medical device 600 may subsequently be secured to a living being at any suitable target location using any of the techniques discussed above.

FIG. 6B shows medical device 600′, which is an embodiment of the present medical devices that is similar to medical device 600, except that mesh element 670 encloses anchoring element 605. Note that “enclosure” does not require that the top and bottom edges of anchoring element 605 be covered, although it does allow for them to be covered. Mesh element 670 includes first mesh element portion 680 that encloses and extends beyond an end (or edge) of first flange 640, and second mesh element portion 690 that encloses and extends beyond an end (or edge) of second flange 660. Mesh element 670 and anchoring element 605 may be coupled to each other using any suitable technique described above.

In each of the embodiments of the present medical devices, including the embodiments set forth above, the anchoring element may be an existing anchoring element known to those of ordinary skill in the art. Example of such anchoring elements are set forth in U.S. Pat. Nos. 6,554,802; 6,937,897; 6,527,587; 6,413,240; 6,358,230; 6,231,548; 5,833,667; 5,814,021; and 5,800,402, each of which are each specifically incorporated by reference in their entirety. Although not shown in the figures, the present anchoring elements may include one or more corrugations along the length of the anchoring element, where the one or more corrugations are configured to provide for the additional placement of one or more sutures to secure the medical devices.

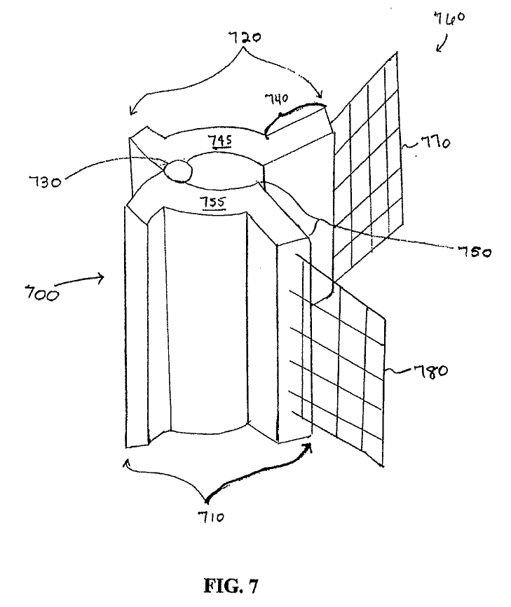

FIG. 7 depicts medical device 700, which is another embodiment of the present medical devices. Medical device 700 includes anchoring element 705, which is coupled to mesh element 760. Anchoring element 705 includes first jaw portion 710 and second jaw portion 720, which are coupled to each other by connecting element 730. Connecting element 730 can be any connecting element known to those of ordinary skill in the art, such as a hinge. Although the embodiment of anchoring element 705 includes two rear portions that protrude beyond connecting element 730, other embodiments lack such rear portions. Anchoring element 705 comprises a medical element-retaining section that includes the two curved portions of the jaw portions. First jaw portion 710 includes a first curved portion 745 from which first flange 740 extends, and second jaw portion 720 includes a second curved portion 755 from which second flange 750 extends. Mesh element 760 includes a first mesh element portion 770 coupled to first flange 740 in any manner described above, and second mesh element portion 780, which may be coupled to second flange 750 in any manner described above.

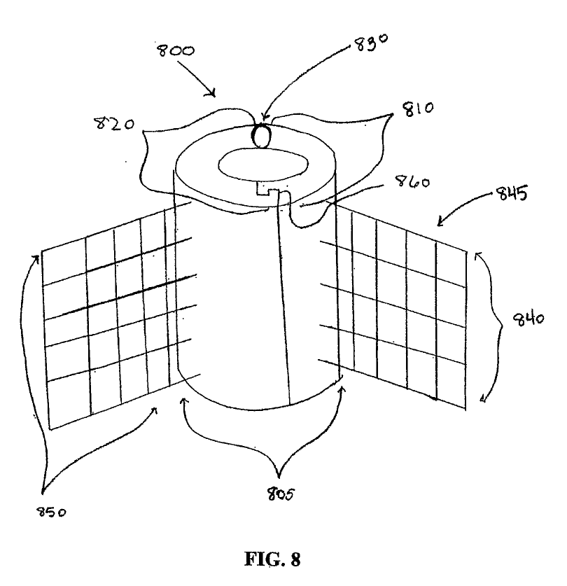

FIG. 8 depicts medical device 800, which is another embodiment of the present medical devices. Medical device 800 includes anchoring element 805, which is coupled to mesh element 845. Anchoring element 805 includes a first jaw portion 810 and second jaw portion 820, which are connected to each other by a connecting element 830. Mesh element 845 includes a first mesh element portion 840 that is coupled to and that extends from first jaw portion 810, and a second mesh element portion 850 that is coupled to and that extends from second jaw portion 820. In this embodiment, anchoring element 805 is configured (and, more specifically, the first and second jaw portions are each configured) such that first jaw portion 810 and second jaw portion 820 can be interlocked to each other as indicated at 860. The interlocking configuration of the jaw portions may be any suitable configuration known to those of ordinary skill in the art.

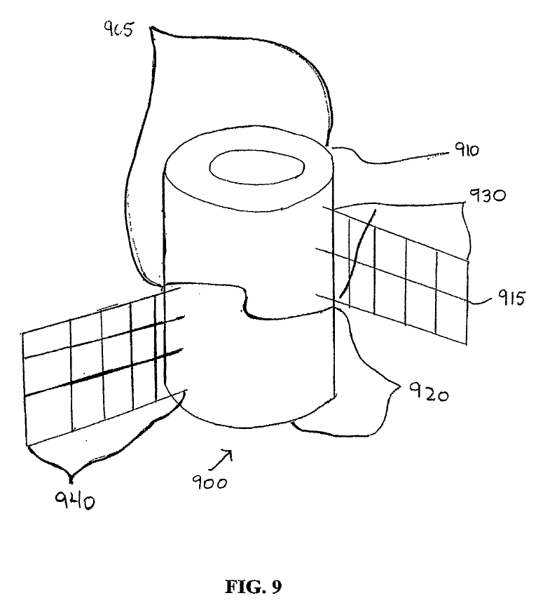

FIG. 9 depicts medical device 900, another embodiment of the present medical devices in which the anchoring element comprises multiple pieces. Medical device 900 includes anchoring element 905, which is coupled to mesh element 915. Anchoring element 905 includes first anchoring element portion 910 and second anchoring element portion 920, which are configured to be coupled together using any suitable attachment style known to those of ordinary skill in the art, such as interlocking parts, screws, a latch, etc. Mesh element 915 includes first mesh element portion 930 and second mesh element portion 940. First mesh element portion 930 is coupled to and extends from first anchoring element portion 910, and second mesh element portion 940 is coupled to and extends from second portion 920. The mesh element portions may be coupled to the anchoring element portions in any of the ways described above.

Kits

The present kits may include an anchoring element and a mesh element coupled to the anchoring element, a tube or wire, and instructions for use, in suitable packaging. In some embodiments, the tube is a flexible tube, such as a catheter, a chest tube, or a urological catheter. In certain particular embodiments, the catheter is an intrathecal catheter. In certain other embodiments, the wire is a stimulator lead or a pacemaker electrode.

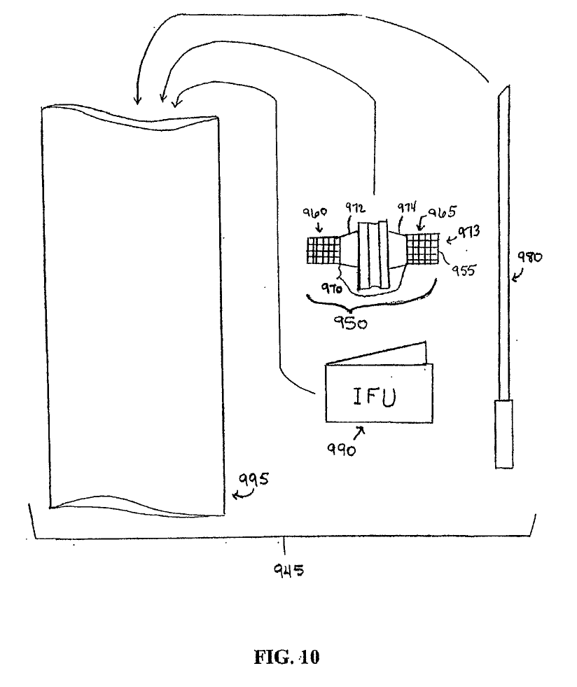

FIG. 10 shows one such kit: kit 945. Kit 945 includes medical device 950 that includes anchoring element 970, which is coupled to mesh element 973. Anchoring element 970 comprises a medical element-retaining section to which first flange 972 and second flange 974 are coupled (e.g., integrally). Mesh element 973 includes first mesh element portion 960, which may be coupled to first flange 972 in any of the ways set forth above, and second mesh element portion 965, which may be coupled to second flange 974 in any of the ways set forth above. Kit 945 also includes catheter 980, instructions for use (IFU) 990, and packaging 995. Packaging 995 is of sufficient size and shape to accommodate medical device 950, catheter 980, and IFU 990. In particular embodiments, the packaging is sealed such that its presterilized contents can be maintained sterile. Sterilization and sealing of packaging can be by any method known to those of ordinary skill in the art.

The present medical devices may be sterilized and sold in groups (such as in blister packs) or individually in sealed plastic bags, as is typical of fittings designed for a single, sterile use.

Methods

The present methods include any procedure that involves securing a medical element—such as a catheter or wire—to a living being using a medical device that comprises a mesh element that is coupled to an anchoring element. The present methods also include methods of delivering a drug to a subject that involve contacting the subject with a medical device of the present invention, where a drug is in contact with the mesh element of the medical device. The subject can be any subject, such as a primate or a laboratory animal. In particular embodiments, the subject is a human, such as a human with chronic pain.

Any method known to those of ordinary skill in the art can be used to secure the medical device to tissue. The securing may take the form of stapling the medical device to suitable tissue, such as muscle or deep fascia if the target location is subcutaneous, or skin. Procedures that require a surgical incision, such as interventions designed to reduce pain using an intrathecal catheter with a pump system or a spinal cord stimulator system, may take less time using the present methods and medical devices when no suturing is involved, and an attachment approach such as stapling is utilized, especially with a staple gun.

The present medical devices, kits and methods are not intended to be limited to the particular forms disclosed. Rather, they are to cover all modifications, equivalents, and alternatives falling within the scope of the claims. For example, although the mesh elements depicted in FIGS. 1-10 are rectangular in shape, they could alternatively extend outwardly and away from the anchoring element to a point (e.g., be triangular in shape). As another example, although the flange elements depicted in FIGS. 3, 5, 7, and 10 are flat, they could just as easily be curved. As a further example, the kit in FIG. 10 could alternatively include a disposable stapling device. Furthermore, in some embodiments it may be advantageous to coat certain surfaces of the medical devices. For example, a coating could be placed on any suitable mesh element. Any suitable coating could be used, such as one that tends to thwart infection or otherwise make the medical device more acceptable to a living being.

The claims are not to be interpreted as including means-plus- or step-plus-function limitations, unless such a limitation is explicitly recited in a given claim using the phrase(s) “means for” or “step for,” respectively.

1. A medical device comprising a rigid anchoring element and a mesh element coupled to the rigid anchoring element.

2. The medical device of claim 1, where the rigid anchoring element comprises a medical element-retaining section.

3-6. (canceled)

7. The medical device of claim 2, where the medical element-retaining section has two ends and is open at each end.

8. The medical device of claim 2, where the rigid anchoring element further comprises a first flange coupled to the medical element-retaining section.

9. (canceled)

10. The medical device of claim 8, where rigid anchoring element further comprises a second flange coupled to the medical element-retaining section.

11. (canceled)

12. The medical device of claim 1, where the mesh element encloses the rigid anchoring element.

13. The medical device of claim 10, where the mesh element comprises a first mesh element portion attached to the first flange and a second mesh element portion attached to the second flange.

14. The medical device of claim 10, where the mesh element encloses the rigid anchoring element.

15. The medical device of claim 10, where the mesh element comprises a shell coupled to an outer surface of the rigid anchoring element, the mesh element including a first portion that extends beyond an end of the first flange and a second portion that extends beyond an end of the second flange.

16-17. (canceled)

18. The medical device of claim 1, where the mesh element comprises a plurality of interwoven filaments.

19-29. (canceled)

30. The medical device of claim 1, where the rigid anchoring element comprises a first jaw portion and a second jaw portion connected to each other.

31. The medical device of claim 30, where the mesh element extends from the first jaw portion or the second jaw portion.

32. (canceled)

33. The medical device of claim 30, where the first jaw portion is hinged to the second jaw portion at one location.

34. (canceled)

35. The medical device of claim 1, where the medical device further comprises a drug molecule attached to the mesh element.

36-42. (canceled)

43. A medical device comprising:
a first jaw element;
a second jaw element connected to the first jaw element;
a first mesh element coupled to the first jaw element; and
a second mesh element coupled to the second jaw element.

44. The medical device of claim 43, where the second jaw element is hinged to the first jaw element.

45. A medical device comprising a rigid anchoring element and a mesh element coupled to the rigid anchoring element, the medical device being configured such that a linear portion of a medical element can be:
at least partially surrounded by the rigid anchoring element;
positioned between the rigid anchoring element and tissue; and
secured to tissue using one or more staples that penetrate the mesh element.

46. The medical device of claim 45, where the rigid anchoring element comprises a medical element-retaining section.

47-49. (canceled)

50. The medical device of claim 45, where the rigid anchoring element comprises a first flange coupled to the medical element-retaining section.

51. (canceled)

52. The medical device of claim 50, where the rigid anchoring element comprises a second flange coupled to the medical element retaining section.

53. (canceled)

54. The medical device of claim 45, where the mesh element encloses the rigid anchoring element.

55. (canceled)

56. The medical device of claim 52, where the mesh element encloses the rigid anchoring element.

57. The medical device of claim 52, where the mesh element comprises a shell coupled to an outer surface of the rigid anchoring element, the mesh element including a first portion that extends beyond an end of the first flange and a second portion that extends beyond an end of the second flange.

58. The medical device of claim 45, where the rigid anchoring element comprises a first jaw portion and a second jaw portion connected to each other.

59. The medical device of claim 58, where the mesh element extends from the first jaw portion or the second jaw portion.

60. (canceled)

61. The medical device of claim 58, where the first jaw portion is hinged to the second jaw portion at one location.

62-63. (canceled)

64. A kit comprising:
a) a medical device comprising a rigid anchoring element and a mesh element coupled to the anchoring element; and
b) a tube or wire.

65. The kit of claim 64, where the tube is a catheter, a chest tube, or a urological catheter.

66. The kit of claim 65, where the tube is a catheter.

67-68. (canceled)

69. The kit of claim 64, where the medical device comprises a drug attached to the mesh element.

70-84. (canceled)
